(19) United States
(12) Patent Application Publication
(10) Pub. No.: US 2021/0008296 A1
(43) Pub. Date: Jan. 14, 2021

(54) INJECTION DEVICE WITH MEANS FOR DETERMINING EXPELLED DOSE

(71) Applicant: NOVO NORDISK A/S, Bagsvaerd (DK)

(72) Inventor: Nikolaj Eusebius JAKOBSEN, Soeborg (DK)

(73) Assignee: Novo Nordisk A/S, Bagsvaerd (DK)

(21) Appl. No.: 16/646,886

(22) PCT Filed: Sep. 14, 2018
(86) PCT No.: PCT/EP2018/074853

§ 371 Date: Mar. 12, 2020

(30) Foreign Application Priority Data
Sep. 14, 2017 (EP) 17191176.1

(51) Int. Cl. A61M 5/315; G16H 20/17

(52) U.S. Cl. CPC A61M 5/31586; A61M 5/31541; A61M 5/31511; A61M 5/31526; A61M 5/31568; A61M 2205/82; G16H 20/17; A61M 2205/50; A61M 2205/3553; A61M 2205/332; A61M 2205/3331; A61M 5/31553

(57) ABSTRACT

The present invention provides an injection device (1, 101, 201) comprising a housing (2, 102, 202), a cartridge (30, 130, 230) holding a medical substance and comprising an outlet and a piston (31, 131, 231), a dose expelling mechanism comprising a piston rod system (10, 50, 110, 150, 210, 250) adapted to be moved relative to the housing (2, 102, 202) during a dose expelling action to thereby advance the piston (31, 131, 231) in the cartridge (30, 130, 230), and a ratchet arm (12) operatively coupled with the piston rod system (10, 50, 110, 150, 210, 250) and configured to undergo a deflecting motion relative to the housing (2, 102, 202) during a particular movement of the piston rod system (10, 50, 110, 150, 210, 250) which corresponds to a predetermined volume of the medical substance being expelled from the cartridge (30, 130, 230), the deflecting motion comprising a first part motion which decelerates the piston rod system (10, 50, 110, 150, 210, 250) followed by a second part motion which accelerates the piston rod system (10, 50, 110, 150, 210, 250), an integrated sensor (76, 176, 276) arranged to detect occurrences of acceleration of the piston rod system (10, 50, 110, 150, 210, 250), and a processor (75, 175, 275) configured to register the occurrences detected by the integrated sensor (76, 176, 276) during the dose expelling action.

### FIELD OF THE INVENTION

The present invention relates generally to drug delivery devices and more specifically to medical injection devices having means for determining the size of an expelled dose.

### BACKGROUND OF THE INVENTION

In the diabetes care segment parenteral drug administration carried out using a traditional vial and syringe system is increasingly being substituted by administration using a pen injection device. Pen injection devices are particularly convenient in that they allow the user to perform a dosed injection from a prefilled drug reservoir without first having to manually transfer the particular dose from one reservoir (the vial) to another (the syringe).

Predominantly, two types of pen injection devices are available, durable injection devices being capable of delivering one or more doses of drug from a prefilled drug cartridge which can be loaded into the device before use and replaced after exhaustion, and disposable injection devices being capable of delivering one or more doses of drug from a prefilled and non-exchangeable drug cartridge. Each of these types of pen injection devices are, or may in principle be, realised in various sub-types, such as e.g. single shot devices adapted to deliver only one dose from a drug cartridge, multi-shot devices capable of delivering a plurality of doses from a drug cartridge, manual devices, where the user provides the force needed for injection, automatic devices having a built-in energy source releasable to occasion the injection, fixed dose devices adapted to deliver the same predetermined dose of drug at each injection event, variable dose devices offering delivery of different doses of drug, settable by the user, etc.

As the labels suggest a durable injection device is intended for use over a considerable period of time during which multiple drug cartridges are exhausted and replaced, whereas a disposable injection device is intended for use until its dedicated drug cartridge is exhausted, after which the entire injection device is discarded.

In the treatment of diabetes it is advisable to keep a log of the administered doses of a particular drug (e.g. insulin or glp-1), including the respective times of dose administration. Some injection devices accordingly offer electronic dose capturing and the opportunity to review dose related information on a digital display.

As an example, U.S. Pat. No. 6,277,099 B1 (Becton, Dickinson and Company) discloses an electronic medication delivery pen, wherein a dialled dose is detected by a piezoelectric sensor arrangement, activated in response to rotation of a user manipulable dose knob, and displayed on a liquid crystal display. The medication delivery pen also comprises a memory function, which together with the liquid crystal display provides an operable interface for conveying the dose size and the time of the last five injections.

However, such type of construction is relatively expensive and not economically viable as a disposable injection device solution.

Marketed disposable injection pens such as FlexPen® and FlexTouch® by Novo Nordisk A/S offer verification of an on-going dose delivery in the form of audible clicks produced by ratchet arms in response to movement of a piston rod drive element. In these injection pens each such click reflects a single unit of medicament expelled from the reservoir.

WO 2007/107564 (Novo Nordisk A/S) discloses an external add-on module for attachment to a pen injection device, which add-on module comprises a miniature microphone capable of picking up mechanical click sounds. The add-on module is adapted to be attached to an exterior housing surface of the pen injection device for detection of clicks produced by the dose expelling mechanism. By counting the number of clicks detected the size of the expelled dose can be determined.

While the above solution enables an automatic registration of the expelled dose it does require an additional component in the system which the user must handle and which when attached to the pen injection device leads to a noticeable asymmetry in terms of both physical appearance and weight distribution that some users consider undesirable.

### SUMMARY OF THE INVENTION

It is an object of the invention to eliminate or reduce at least one drawback of the prior art, or to provide a useful alternative to prior art solutions.

In particular, it is an object of the invention to provide a solution for automatic registration of an expelled dose of drug which is sufficiently inexpensive to enable a cost-effective implementation in a disposable injection device.

It is a further object of the invention to provide such a solution which requires a minimum of user handling and which renders a symmetric configuration of the injection device possible.

It is also an object of the invention to provide an injection device having means for automatic registration of an expelled dose which is precise and reliable.

In the disclosure of the present invention, aspects and embodiments will be described which will address one or more of the above objects and/or which will address objects apparent from the following text.

In one aspect of the invention an injection device according to claim 1 is provided.

Hence, an injection device is provided which comprises 1) a housing, 2) a cartridge holding a medical substance and comprising an outlet, e.g. sealed by a penetrable septum such as a self-sealing rubber diaphragm, and a piston, and 3) a dose expelling mechanism. The dose expelling mechanism comprises a piston rod system adapted to be moved relative to the housing during a dose expelling action to thereby advance the piston in the cartridge, and a ratchet arm operatively coupled with the piston rod system and configured to undergo a deflecting motion relative to the housing during a particular movement of the piston rod system which corresponds to a predetermined volume of substance, e.g. one increment or unit, being expelled from the cartridge. The deflecting motion comprises a first part motion momentarily decelerating the piston rod system followed by a second part motion momentarily accelerating the piston rod system. The injection device further comprises an integrated sensor adapted to detect occurrences of acceleration of the piston rod system, and a processor configured to register each occurrence of acceleration of the piston rod system detected by the integrated sensor during the dose expelling action. The processor may also be integrated in the injection device, e.g. in the vicinity of the integrated sensor.

Since one deflecting motion of the ratchet arm is correlated with the delivery of a predetermined volume of substance and one deflecting motion of the ratchet arm involves one occurrence of acceleration of the piston rod system, the above solution enables a determination of an expelled dose by summation of occurrences detected by the integrated sensor.

In the present context, an “integrated” component is a component which is positioned within the injection device and which therefore is inaccessible to the user.

The integrated sensor prevents a change of lateral weight distribution of the injection device as no physical entity needs to be attached to the exterior of the housing for a dose logging to take place. Furthermore, in case the injection device is of the conventional pen-shaped type the integrated sensor enables preservation of the axisymmetric, or near axisymmetric, exterior. The detection of accelerations of the piston rod system provides for an accurate and reliable dose determination which can be realised by use of an inexpensive sensor system.

The processor may further be configured to calculate a sum of the occurrences of acceleration of the piston rod system detected by the integrated sensor during the dose expelling action, whereby a dose determination is completed in the injection device itself. A memory device may be included in the injection device for storage of determined doses.

If the injection device further comprises an electronic display, said display may be configured to show the result of the dose determination. Regardless, the injection device may further comprise wireless communication means for transferring information regarding the occurrences of acceleration of the piston rod system detected by the integrated sensor during the dose expelling action to an external data receiving device. The transferred information may either be processed, e.g. in the form of the calculated sum representing the expelled dose, or raw in the form of each and every occurrence registered by the processor, in which case the dose determination may be carried out in the data receiving device.

The data receiving device may for example be a mobile processing unit such as a mobile phone, a tablet, or a portable pc, another medical device such as e.g. a body substance measuring device or a drug delivery device, a network operated or connected device, or any other suitable electronic device.

The piston rod system may comprise a piston rod extending along a longitudinal axis from a proximal piston rod end to a distal piston rod end, and a piston washer being arranged at the distal piston rod end. During use of the injection device, the piston washer will thereby be arranged between the distal piston rod end and the piston. The piston rod system may be manually activated, e.g. by the user depressing an injection button a distance correlated with the dose to be expelled, automatically activated, e.g. by release of energy from an energy source such as a spring member, e.g. a torsion spring or a compression spring, operatively coupled with the piston rod, or semi-automatically activated by combination of a manually applied force and release of energy from an energy source.

The deflecting motion may be prompted by interaction between the ratchet arm and the piston rod or by interaction between the ratchet arm and a piston rod drive element during movement of the piston rod system in connection with a dose expelling action. This may for example be the case if the ratchet arm is arranged on an interior surface of the housing. Alternatively, the deflecting motion may be prompted by interaction between the ratchet arm and a toothed structure on an interior circumferential surface of the housing during movement of the piston rod system in connection with a dose expelling action. This may for example be the case if the ratchet arm is arranged on the piston rod or on a piston rod drive element. The first part motion may be a departing motion away from a base position of the ratchet arm, while the second part motion may be a returning motion towards the base position. In particular, the first part motion may cause elastic energy to be stored in the ratchet arm, and the second part motion may cause stored elastic energy to be released from the ratchet arm. Due to the ratchet arm and the piston rod system being operatively coupled, during storage of elastic energy in the ratchet arm the piston rod system will lose kinetic energy and exhibit deceleration, whereas during release of elastic energy from the ratchet arm the piston rod system will gain kinetic energy and exhibit acceleration.

The acceleration of the piston rod system may in principle be detected in several ways, either directly by optically monitoring the movement of the piston rod system relative to the housing or indirectly by detecting an effect of the accelerating piston rod system. The latter serves as the basis for the present invention and enables the provision of an inexpensive dose logging system.

One effect of the repeated deceleration and acceleration of the piston rod system during a dose expelling action is an irregular force transmission to the piston. Specifically, during the first part motion a portion of the energy imparted to move the piston rod is transferred to the ratchet arm and stored as elastic energy therein, and during the second part motion the stored energy is released from the ratchet arm and transferred back to the piston rod. As a result of this happening repeatedly during expelling of a dose the size of multiple predetermined volumes, e.g. during expelling of a dose of more than one unit, the piston rod provides, on a small scale, a pulsating drive force.

A pulsating movement of the piston rod propagates both upstream and downstream in the sense that it manifests itself in various other parts of the injection device. The integrated sensor may therefore for example be or comprise a force sensor incorporated in a manually actuated injection button arranged at a proximal end of the injection device, or a pressure sensor arranged in a drug containing chamber of the cartridge delimited by the piston, the outlet and an interior side wall portion of the cartridge.

Alternatively, however, the integrated sensor may advantageously be incorporated in the piston washer, as this provides for a particularly large signal output and thereby for a particularly reliable dose determination.

For example, the integrated sensor may be a force sensor arranged to measure the force applied to the piston washer by the piston rod. The force sensor may be a strain or stress responsive sensor such as e.g. a strain gauge or a piezoelectric material. A piezoelectric sensor is attractive because it provides a current output each time it experiences a rapid force change and therefore does not need to be monitored. A piezoelectric sensor solution thus requires less power than e.g. a strain gauge based sensor solution. With a piezoelectric sensor, for example, each acceleration of the piston rod will cause a change in the force applied to the piston washer and thereby generation of a signal by the sensor, which signal is then registered by the processor.

The piezoelectric material may be printed on a flexible substrate, thereby producing a very low cost sensor which is economically more attractive than e.g. a ceramic piezo material on a metal substrate.

The piston washer may comprise a piston rod bearing structure having an interface sheet which is adapted to receive the distal piston rod end, said interface sheet extending transversally to the longitudinal axis and exhibiting axial resilience, and the force sensor may be arranged on the interface sheet. Thereby, the force sensor may be directly excited by the pulsating force applicator when the interface sheet is axially deflected in response to the drive force.

The interface sheet may have a first bending stiffness, and the piston rod bearing structure may further comprise an annular spacer and a supporting plate having a predetermined second bending stiffness which is larger than the first bending stiffness. The supporting plate may be sandwiched between the annular spacer and the interface sheet, and the distal piston rod end may be arranged to abut a portion of the interface sheet that is supported by the supporting plate but unsupported by the annular spacer. The portion of the interface sheet which is in contact with the piston rod is thus allowed to deflect out of its own plane. The role of the supporting plate is to prevent a full and lasting deformation of a flexible interface sheet, as this would prevent a detection of the small force fluctuations. Alternatively, the interface sheet itself may have a bending stiffness comparable to the predetermined second bending stiffness in which case the supporting plate is not needed.

The piston washer may further comprise an electric power supply element, e.g. a battery, arranged to support the annular spacer, and the interface sheet may form part of a flexible foil member which also comprises a bottom sheet arranged in contact with a surface of the electric power supply element opposite the annular spacer, and a connecting portion connecting the interface sheet and the bottom sheet. The foil member may further carry the processor and may comprise electric leads, e.g. printed electric leads, connecting the strain responsive sensor and the processor. In that case the piston washer is a fully self-contained sensor system which can be implemented in the injection device to determine the doses expelled therefrom.

In particular embodiments of the invention the force sensor comprises a piezoelectric material printed on a surface portion of the interface sheet which faces the supporting plate. Thereby, physical contact between the piezoelectric material and the piston rod is avoided, and the risk of mechanical wear of the sensor is eliminated.

As mentioned above the effect of the pulsating movement of the piston rod is also detectable elsewhere in the injection device. Consequently, the integrated sensor may alternatively be a pressure sensor arranged in fluid communication with the medical substance or with a fluid filled hollow of the piston.

In relation to a pressure sensor arranged in fluid communication with the medical substance the piston washer may comprise a hollow structure arranged to extend through the piston and into an interior of the cartridge, and the pressure sensor may be arranged in fluid communication with an interior of the hollow structure. The pressure sensor is thereby capable of detecting pressure fluctuations in the medical substance inside the cartridge arising from the jerking advancement of the piston.

In relation to a pressure sensor arranged in fluid communication with a fluid filled hollow of the piston the piston washer may comprise a hollow structure arranged to extend into the fluid filled hollow of the piston, and the pressure sensor may be arranged in fluid communication with an interior of the hollow structure. The interior of the hollow structure may for example be air filled and may be delimited by a portion of a piston wall and a portion of the piston washer, including the hollow structure, in which case the pressure sensor is capable of detecting pressure fluctuations in a closed air chamber adjacent the medical substance in the cartridge.

In either of these two cases, the piston washer may further comprise a piston rod bearing surface adapted to receive the distal piston rod end, a piston interface layer adapted to interact with the piston, a hub member carrying the hollow structure, the hollow structure extending through the piston interface layer, an electric power supply element, e.g. a battery, arranged between the piston rod bearing surface and the hub member, and a foil member comprising a top sheet arranged between the piston rod bearing surface and the electric power supply element, a bottom sheet arranged between the electric power supply element and the hub member and carrying the pressure sensor, and a connecting portion connecting the top sheet and the bottom sheet, the foil member further carrying the processor and comprising electric leads, e.g. printed electric leads, connecting the pressure sensor and the processor.

In another aspect of the invention a piston washer as described in the above for use in an injection device is provided.

A ratchet arm as described in the present text may be or comprise a resilient click generating arm such as those present in the aforementioned FlexPen® and FlexTouch® devices. In exemplary embodiments of the invention the dose expelling mechanism includes at least two ratchet arms.

For the avoidance of any doubt, in the present context the term “medical substance” designates a medium which is used in the treatment, prevention or diagnosis of a condition, i.e. including a medium having a therapeutic or metabolic effect in the body. Further, the terms “distal” and “proximal” denote positions at or directions along a drug delivery device, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a perspective longitudinal section view of an injection device according to a first embodiment of the invention,

FIG. 2 is a perspective cross-sectional view of a proximal portion of the injection device,

FIG. 3 is a graphical representation of the piston net force during a dose expelling action,

FIG. 4 is an exploded view of a piston washer carrying a force sensor, as used in the injection device,

FIG. 5 is a perspective longitudinal section view of the piston washer,

FIG. 6 is a top view of a flexible substrate carrying printed electronics,

FIG. 7 is a perspective longitudinal section view of a distal portion of an injection device according to a second embodiment of the invention,

FIG. 8 is a perspective longitudinal section view of a piston washer used in the injection device of FIG. 7,

FIG. 9 is a perspective longitudinal section view of a distal portion of an injection device according to a third embodiment of the invention, and

FIG. 10 is a perspective longitudinal section view of a piston washer/piston assembly used in the injection device of FIG. 9.

In the figures like structures are mainly identified by like reference numerals.

### DESCRIPTION OF EXEMPLARY EMBODIMENTS

When in the following relative expressions, such as “upwardly” and “downwardly”, are used, these refer to the appended figures and not necessarily to an actual situation of use. The shown figures are schematic representations for which reason the configuration of the different structures as well as their relative dimensions are intended to serve illustrative purposes only.

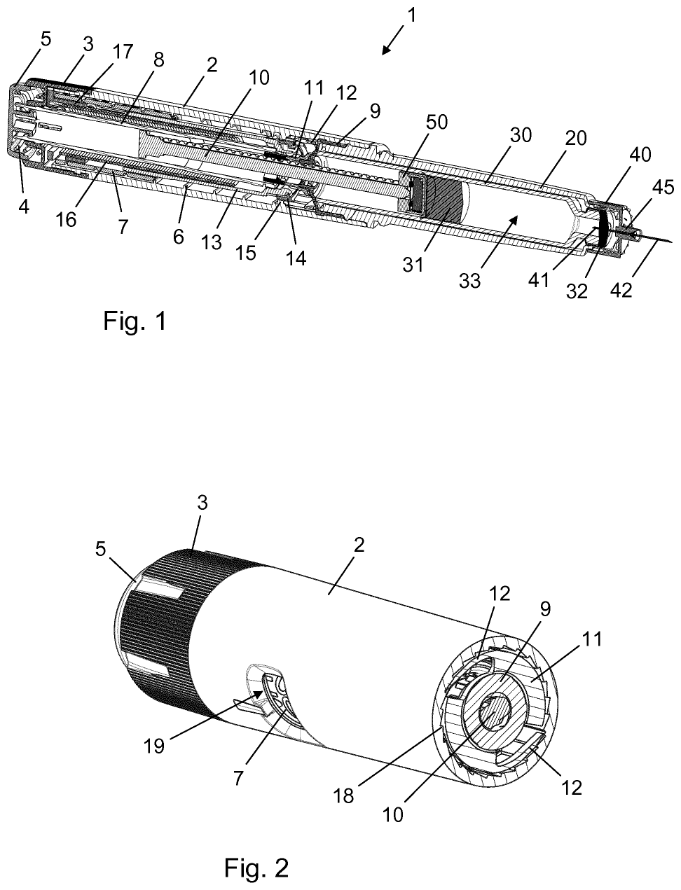

FIG. 1 is a perspective longitudinal section view of an injection pen 1 according to a first embodiment of the invention. The injection pen 1 comprises a housing 2 extending along a longitudinal housing axis and a cartridge holder 20 which is at least axially fixed with respect to the housing 2 and which holds a drug cartridge 30. A nut element 9 is arranged in the housing 2 just proximally of the cartridge holder 20. The nut element 9 serves to support an axially extending piston rod 10 and to enable helical advancement of the piston rod 10 relative to the housing 2 via a threaded interface. The drug cartridge 30 has a generally cylindrical wall which extends between a proximal end and a distal end and which comprises a narrowed distal end portion. The distal end is sealed by a penetrable septum 32 and the drug cartridge 30 further comprises a piston 31 arranged in sealing contact with an interior surface of the generally cylindrical wall such that a chamber 33 is defined by the generally cylindrical wall, the piston 31 and the septum 32. The chamber 33 holds a medical substance. In the present embodiment the injection pen 1 is of the disposable type, and it is not possible to remove the drug cartridge 30 without damaging the injection pen 1. However, it is noted that the injection pen 1 could just as well be a durable type of device allowing for exchange of the drug cartridge 30.

In FIG. 1 an injection needle unit 40 is attached to the injection pen 1. The injection needle unit 40 comprises a needle hub 45 holding an injection needle which comprises a back needle portion 41 extending proximally from the needle hub 45 and a front needle portion 42 extending distally from the needle hub 45. In the present case the back needle portion 41 and the front needle portion 42 form part of a single injection needle element. Alternatively, however, the back needle portion 41 and the front needle portion 42 could be two separate needle elements fluidly connected in the needle hub 45. When the injection needle unit 40 is attached to the injection pen 1 a segment of the back needle portion 41 resides in the chamber 33, following penetration of the septum 32, thereby providing an exit route for the medical substance.

The injection pen 1 is operable to set a desired dose of the medical substance to be injected and to expel the set dose through the injection needle. Accordingly, the injection pen 1 comprises a dose setting mechanism and a dose expelling mechanism. The dose setting mechanism comprises a user operable dose dial 3, a scale drum 7 having a plurality of dose numerals arranged thereon, a reset tube 8, a ratchet tube 13, and a torsion spring 16, and is configured to allow both dialling up and dialling down to set a dose and to adjust a set dose. The particular operation of the dose setting mechanism is similar to the operation of the dose setting system in the injection device disclosed in WO 2015/071354 and will not be described further in the present text, since the dose setting mechanism as such is irrelevant to the present invention, being concerned only with the determination of an expelled dose. For details on the operation of the dose setting mechanism reference is made to the aforementioned WO 2015/071354, particularly p. 10, I. 21-p. 15, I. 13.

In the following the various components, and the operation, of the injection pen 1 will be described based on the dose expelling functionality.

An injection button 5 is slidably arranged at the proximal end of the housing 2. The injection button 5 is axially fixed to the reset tube 8 and is biased proximally by a button spring 4. The reset tube 8 is at its distal end portion axially and rotationally coupled with the ratchet tube 13 such that a distal displacement of the reset tube 8 causes a corresponding distal displacement of the ratchet tube 13 and a rotation of the ratchet tube 13 in a dose expelling direction causes a corresponding rotation of the reset tube 8.

The torsion spring 16 extends axially along an exterior surface of the reset tube 8 and has a proximal end attached to a spring base 17 and a distal end attached to the ratchet tube 13. The spring base 17 is axially and rotationally fixed to the housing 2, and the torsion spring 16 is pre-strained during assembly of the injection pen 1, biasing the ratchet tube 13 in the dose expelling direction relative to the housing 2 (clockwise when seen from the distal end), to ensure sufficient power to expel an entire set dose regardless of its size.

The ratchet tube 13 is rotationally interlocked with the scale drum 7 via a spline interface, and the scale drum 7 is provided with an exterior helical groove which is in engagement with a helical rib 6 on an interior surface portion of the housing 2 such that a rotation of the ratchet tube 13 in the dose expelling direction causes a helical proximal displacement of the scale drum 7 in the housing 2, and a rotation of the ratchet tube 13 opposite the dose expelling direction causes a helical distal displacement of the scale drum 7 in the housing 2.

The ratchet tube 13 is at its distal end portion axially locked to a clutch 14. The clutch 14 is provided with a plurality of exterior spline elements (not visible) which in a dose setting axial position of the clutch 14 engage with corresponding housing splines 15 on an interior surface of the housing 2, thereby rotationally locking the clutch 14 to the housing 2. The clutch 14 is further provided with an interior toothed structure (not visible) configured for interaction with a flexible arm (not visible) on the ratchet tube 13 so as to ensure joint rotation of the ratchet tube 13 and the clutch 14 in the dose expelling direction.

Also, the clutch 14 is rotationally locked to a piston rod drive element 11 arranged about the piston rod 10. The piston rod 10 has an exterior threaded section and two opposite longitudinal grooves (not visible), and the piston rod drive element 11 has a central bore with two opposite protrusions (not visible), each of which engage one of the grooves to provide a rotational interlocking connection between the piston rod drive element 11 and the piston rod 10. The piston rod drive element 11 further has a pair of opposite ratchet arms 12 acting to restrict its rotational movement relative to the housing 2, as explained below in relation to FIG. 2.

During setting of a dose the torsion spring 16 becomes further strained. In order to expel a set dose the injection button 5 is depressed against the proximal end of the housing 2. This will displace the reset tube 8 axially in the distal direction, slaving the ratchet tube 13 and the clutch 14. As a result the clutch 14 will slide out of engagement with the housing splines 15 and begin to rotate in the dose expelling direction driven by the thereby released torsion spring 16 via its rotational connection to the ratchet tube 13.

The rotation of the ratchet tube 13 and the clutch 14 as the torsion spring 16 unwinds causes a helical proximal motion of the scale drum 7 as well as a rotation of the piston rod drive element 11 and, accordingly, of the piston rod 10. Due to the threaded interface between the piston rod 10 and the nut element 9 this will cause a helical distal advancement of the piston rod 10 into the drug cartridge 30. The distal end of the piston rod 10 is connected to a specially designed piston washer 50, described in detail below, which as a result of the movement of the piston rod 10 forces the piston 31 into the drug cartridge 30 to thereby expel the set dose of medical substance from the chamber 33 through the injection needle.

FIG. 2 is a perspective view of a proximal portion of the housing 2, cross-sectioned through the nut element 9 to show that the ratchet arms 12 are axially aligned with ratchet teeth 18 arranged around an interior circumferential surface portion of the housing 2. Each ratchet arm 12 is formed as a circumferential extension of a peripheral portion of the piston rod drive element 11 and thus constitutes a suspended, flexible curved beam having a radially outwardly directed bias. An end portion of each ratchet arm 12 interacts with a section of the ratchet teeth 18, providing a unidirectional ratchet mechanism which prevents counterclockwise (seen from the distal end) rotation of the piston rod drive element 11 relative to the housing 2.

In the course of the dose expelling action described above the joint rotation of the ratchet tube 13, the clutch 14, and the piston rod drive element 11 in the dose expelling direction causes each of the ratchet arms 12 to ride over a number of the ratchet teeth 18. The ratchet mechanism is configured such that two opposite ratchet teeth 18 are passed by the respective ratchet arms 12 simultaneously, and one such simultaneous passage of two opposite ratchet teeth 18 is correlated with one unit of the medical substance being expelled from the drug cartridge 30.

During the clockwise rotation of the piston rod drive element 11, as a consequence of the interaction with the ratchet teeth 18 and their respective directional bias, each of the ratchet arms 12 will undergo a deflecting motion as it passes one of the ratchet teeth 18. Observing one of the ratchet arms 12, an angular displacement of the piston rod drive element 11 corresponding to one unit of the medical substance being expelled from the drug cartridge 30 will cause the end portion of the ratchet arm 12 to firstly slide along a ratchet tooth 18 from a tooth trough base position to a tooth tip deflected position and secondly to pass the tooth tip and assume a new base position at the subsequent tooth trough. In the present context this is referred to as one deflecting motion of the ratchet arm 12, which then comprises a first part motion from the tooth trough base position to the tooth tip deflected position and a second part motion from the tooth tip deflected position to the new base position.

The movement from the tooth trough base position to the tooth tip deflected position deflects the ratchet arm 12 gradually radially inwardly, against its bias, thereby storing energy in the ratchet arm 12 and increasing the friction between the end portion of the ratchet arm 12 and the ratchet tooth 18, resulting in a momentary decrease of the speed of rotation of the piston rod drive element 11. As the end portion of the ratchet arm 12 passes the tooth tip the energy stored in the ratchet arm 12 is released, forcing the end portion of the ratchet arm 12 towards the subsequent tooth trough, and the friction is abruptly reduced, resulting in a momentary increase of the speed of rotation of the piston rod drive element 11.

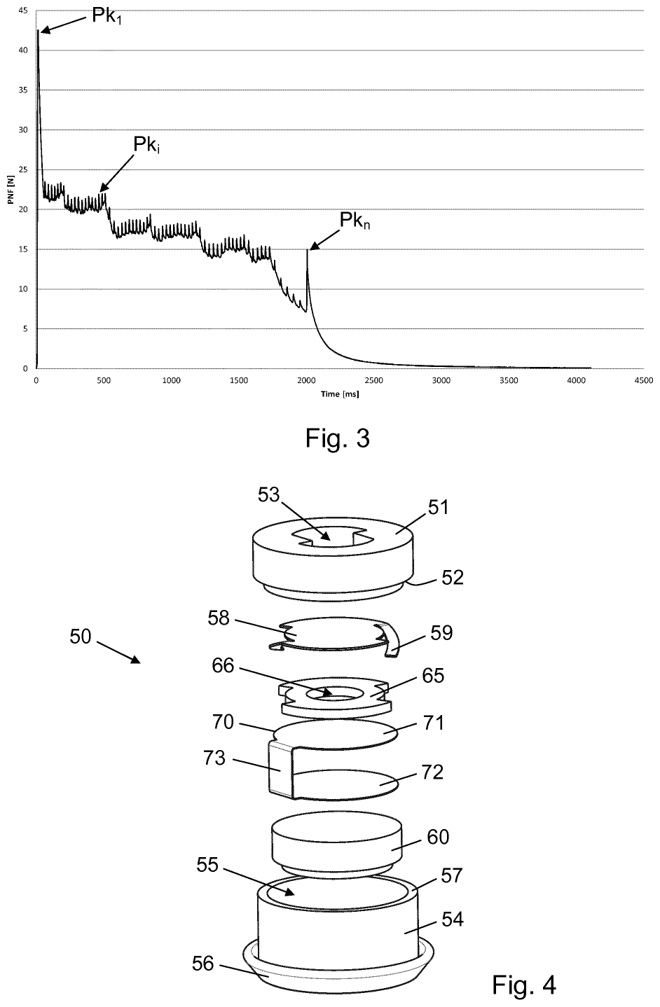

This repetitive accumulation and release of energy is reflected in the piston net force, i.e. the force which the piston rod 10, which is driven by the rotation of the piston rod drive element 11, applies to the piston 31 via the piston washer 50. FIG. 3 illustrates the piston net force variation over time during an expelling of n units of the medical substance from the drug cartridge 30. As can be seen the piston net force fluctuates as the dose expelling progresses, delimited by a first peak, Pk₁, and a last peak, Pkₙ. Each individual peak, Pkᵢ, reflects a deflecting motion of both ratchet arms 12, and since each deflecting motion of both ratchet arms 12 corresponds to one unit of the medical substance expelled from the drug cartridge 30 the total expelled dose can be determined by counting and adding up the peaks, Pkᵢ, exhibited during the dose expelling action.

To that end the injection pen 1 comprises the specially designed piston washer 50. FIG. 4 shows the constituent parts of the piston washer 50 in an exploded view. It generally comprises a top part 51 and a bottom part 54, together forming a container for accommodation of a sensor system which is configured to detect the piston net force fluctuations. The top part 51 provides a lid having a bore 53, shaped to receive and provide a rotationally interlocked connection with a distal end portion of the piston rod 10, and a circumferential overhang 52, the latter serving as an abutment interface for a seat 57 on the bottom part 54. The bottom part 54 comprises a rigid cup-shaped structure defining a cavity 55 and a flexible piston interface layer 56 rotationally locked to the cup-shaped structure, e.g. via 2K moulding. The cavity 55 houses a flexible PCB 70 comprising a top sheet 71, a bottom sheet 72 and a bridge 73, a battery 60 arranged between the top sheet 71 and the bottom sheet 72, a spacer 65 having a central bore 66 and being arranged between the top sheet 71 and the battery 60, and a thin metal plate 58 arranged between the top sheet 71 and the spacer 65. The metal plate 58 has a pair of battery pads 59 providing electrical connection to the battery 60.

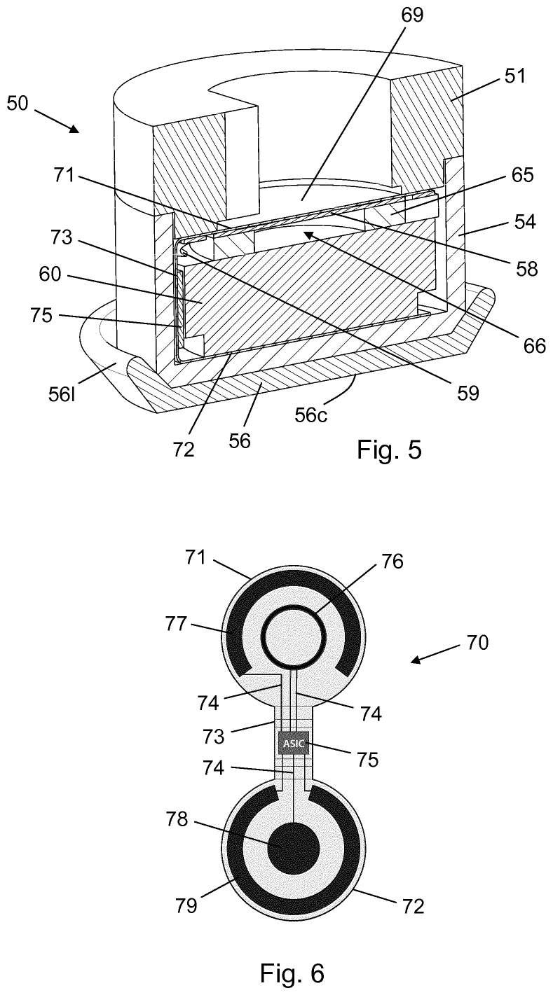

FIG. 5 is a cross-sectional view of the piston washer 50 in an assembled state. The top part 51 and the bottom part 54 are welded together to provide an axially and rotationally interlocked connection which ensures that the piston washer 50 as a whole rotates jointly with the piston rod 10. In other embodiments of the invention the top part 51 and the bottom part 54 may be free to rotate relative to one another and/or the bore 53 may be shaped to allow rotation of the piston rod 10 relative to the top part 51. The piston interface layer 56 comprises a contact surface 56c adapted to abut the piston 31 and a sealing lip 561 configured for sealing contact with an interior wall portion of the drug cartridge 30. A chip 75 is mounted, or alternatively printed, onto the bridge 73 and is thus positioned next to the battery 60.

As can be seen from FIG. 5 a central portion of the metal plate 58 is suspended over the central bore 66 of the spacer 65. A bearing surface 69 adapted to receive the distal end of the piston rod 10 is constituted by an upwardly directed underside of the top sheet 71 supported by the central portion of the metal plate 58 being suspended over the central bore 66. During a dose expelling action of the injection pen 1 the distal end of the piston rod 10 will apply an axial force to the bearing surface 69 which will then be depressed somewhat into the central bore 66. The primary purpose of the metal plate 58 is to prevent a full and lasting deformation of the flexible top sheet 71 as this would render impossible the detection of piston net force fluctuations (explained below).

FIG. 6 is a top view of the flexible PCB 70 in an unwrapped configuration, showing the arrangement of the chip 75 and various printed electronics components thereon. The top sheet 71 carries a piezoelectric sensor 76 and a battery pad 77, and the bottom sheet 72 carries a battery pad 78 and an antenna 79. Electric leads 74 connect the chip 75 to the other components on the flexible PCB 70.

The piezoelectric sensor 76 is sensitive to deflections of the top sheet 71 out of its plane and produces an output which is proportional to the strain in the sensor material. Accordingly, the piezoelectric sensor 76 is suitable for detection of small variations in the force of the piston rod 10 to the bearing surface 69, as realised during a dose expelling action where the deflections of the ratchet arms 12 repeatedly brakes and accelerates the piston rod 10. The presence and configuration of the metal plate 58 provides for small and counteracted deformations of the top sheet 71 in the desired force area which is necessary in order to obtain a repeated activation of the piezoelectric sensor 76. It is noted, however, that in alternative embodiments of the invention the piezoelectric sensor 76 may be arranged on a surface which is itself of sufficient rigidity to provide the desired resistance to deflection caused by the piston rod 10.

In this embodiment, with such counteracting component in place each occurrence of deflecting motion of the ratchet arms 12 is detected by the piezoelectric sensor 76 and registered by the chip 75 which is configured to, following completion of the dose expelling action, calculate a sum of the occurrences detected by the piezoelectric sensor 76 to thereby determine the size of the expelled dose.

The chip 75 is further configured to relay the determined expelled dose size to an exterior device (not shown) via the antenna 79. The exterior device may for example be a mobile processing unit such as a mobile phone, a tablet, or a portable pc, another medical device such as e.g. a body substance measuring device or a drug delivery device, a network operated or connected device, or any other suitable receiving device.

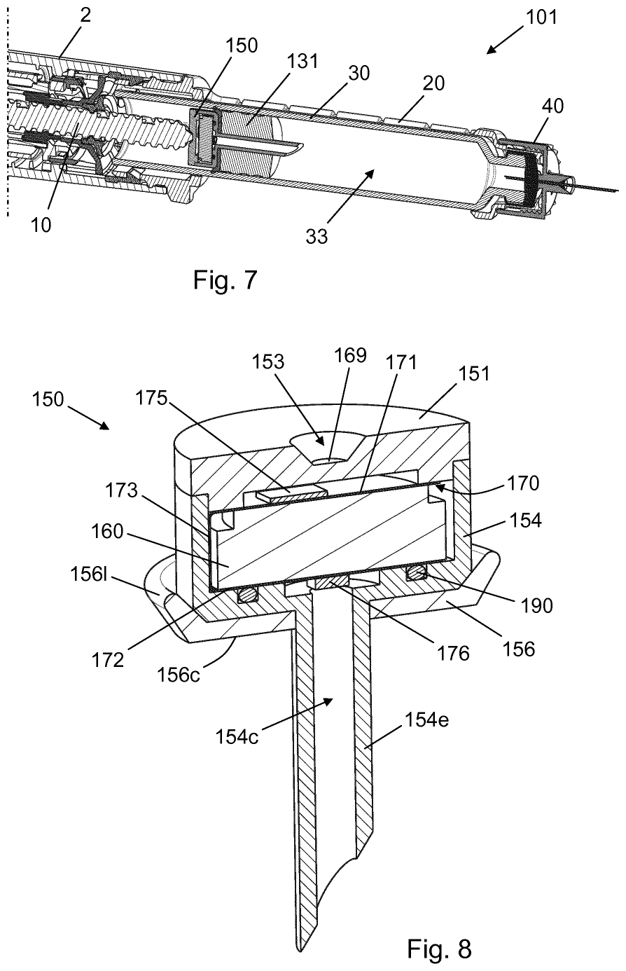

FIG. 7 is a perspective longitudinal section view of a distal portion of an injection pen 101 according to a second embodiment of the invention. The injection pen 101 is functionally and visually similar to the injection pen 1 according to the first embodiment of the invention, but differs therefrom in that it comprises an alternative piston washer 150, an alternative piston 131, and a pressure based sensor system.

FIG. 8 is a cross-sectional view of the piston washer 150 which comprises a top part 151 and a bottom part 154 welded together to provide an axially and rotationally interlocked connection. The top part 151 has a recess 153 for reception of the distal end portion of the piston rod 10 and a bearing surface 169 to which the piston rod 10 applies a driving force during a dose expelling action. The bottom part 154 comprises a piston interface layer 156, having a contact surface 156c adapted to abut the piston 131 and a flexible sealing lip 1561 configured for sealing contact with an interior wall portion of the drug cartridge 30, and a rigid cup shaped structure which defines a cavity, much like that of the bottom part 54 of the piston washer 50 according to the first embodiment of the invention. However the bottom part 154 also has a tubular needle-shaped extension 154e defining a canal 154c which leads into the cavity defined by the cup shaped structure. The extension 154e extends distally through a hole in the piston interface layer 156.

The cavity defined by the cup shaped structure accommodates a battery 160 and a flexible PCB 170 partly wrapped around the battery 160. The flexible PCB 170 comprises a top sheet 171, a bottom sheet 172, and a bridge 173. The top sheet 171 carries a chip 175 and the bottom sheet 172 carries a pressure sensor 176. Furthermore, the flexible PCB 170 comprises printed electronics components (not visible) similar to those on the flexible PCB 70 of the first embodiment of the invention, including electric leads connecting the pressure sensor 176 and the chip 175.

The piston 131 has a through-going bore and is sealingly fitted around the extension 154e. As indicated in FIG. 7 the respective axial dimensions of the piston 131 and the extension 154e are such that a tip of the extension 154e resides in the chamber 33. The pressure sensor 176 is arranged in fluid communication with the canal 154c, whereby during use of the injection pen 101 the pressure sensor 176 is in fluid communication with the chamber 33, enabling a monitoring of the pressure inside the drug cartridge 30. An O-ring 190 is arranged to preserve a dry environment for the battery 160.

During a dose expelling event the above described repeated deceleration and acceleration of the piston rod 10, arising from the deflecting motion of the ratchet arms 12, will cause a step-wise advancement of the piston 131 in the drug cartridge 30. This creates a pulsating pressure in the chamber 33, where each pressure peak corresponds to a unit of dose delivered. The pressure sensor 176 detects the pressure peaks and calculates a sum to thereby determine the size of the expelled dose.

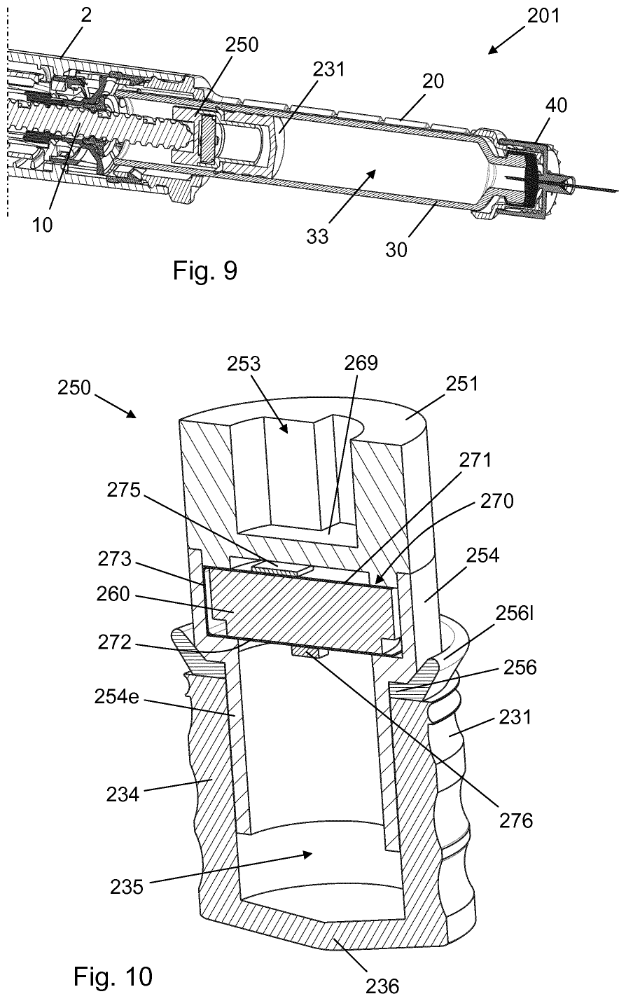

FIG. 9 is a perspective longitudinal section view of a distal portion of an injection pen 201 according to a third embodiment of the invention. The injection pen 201 is functionally and visually similar to the injection pen 1 according to the first embodiment of the invention, but differs therefrom in that it comprises an alternative piston washer 250, an alternative piston 231, and a pressure based sensor system.

FIG. 10 is a cross-sectional view of an assembly of the piston washer 250 and the piston 231. The piston washer 250 comprises a top part 251 and a bottom part 254 welded together to provide an axially and rotationally interlocked connection. The top part 251 has a bore 253, shaped to receive and provide a rotationally interlocked connection with a distal end portion of the piston rod 10, and a bearing surface 269 to which the piston rod 10 applies a driving force during a dose expelling action. The bottom part 254 comprises a piston interface layer 256, having a contact surface adapted to abut the piston 231 and a flexible sealing lip 2561 configured for sealing contact with an interior wall portion of the drug cartridge 30, and a rigid cup shaped structure which defines a cavity. The bottom part 254 resembles the bottom part 154 of the piston washer 150 according to the second embodiment of the invention, but has a tubular extension 254e which is blunt instead of needle-shaped.

The cavity defined by the cup shaped structure accommodates a battery 260 and a flexible PCB 270 partly wrapped around the battery 260. The flexible PCB 270 comprises a top sheet 271, a bottom sheet 272, and a bridge 273. The top sheet 271 carries a chip 275 and the bottom sheet 272 carries a pressure sensor 276. Furthermore, the flexible PCB 270 comprises printed electronics components (not visible) similar to those on the flexible PCB 70 of the first embodiment of the invention, including electric leads connecting the pressure sensor 276 and the chip 275.

The piston 231 is hollowed out and thus comprises a piston cavity 235 defined by a generally cylindrical side wall 234 and a transversal end wall 235. The tubular extension 254e is positioned within the piston cavity 235 in close contact with an interior wall portion of the side wall 234. A confined space is thereby established between the end wall 236, a portion of the side wall 234, the tubular extension 254e, and the bottom sheet 272, and the pressure sensor 276 is arranged in fluid communication with this confined space.

During a dose expelling event the repeated deceleration and acceleration of the piston rod 10, arising from the deflecting motion of the ratchet arms 12, will cause small axial deformations of the piston 231 and, accordingly, small volume changes of the confined space. Each volume reduction of the confined space results in a pressure peak which is detected by the pressure sensor 176. At the end of the dose expelling the pressure sensor 176 calculates a sum of detected pressure peaks and thereby determines the size of the expelled dose.

1. An injection device comprising:
a housing,
a cartridge holding a medical substance and comprising an outlet and a piston,
a dose expelling mechanism comprising:
a piston rod system adapted to be moved relative to the housing during a dose expelling action to thereby advance the piston in the cartridge, and
a ratchet arm operatively coupled with the piston rod system and configured to undergo a deflecting motion relative to the housing during a particular movement of the piston rod system which corresponds to a predetermined volume of the medical substance being expelled from the cartridge, the deflecting motion comprising a first part motion which decelerates the piston rod system followed by a second part motion which accelerates the piston rod system,
an integrated sensor arranged to detect occurrences of acceleration of the piston rod system, and
a processor configured to register the occurrences detected by the integrated sensor during the dose expelling action.

2. The injection device according to claim 1, wherein the processor is further configured to calculate a sum of the occurrences detected by the integrated sensor during the dose expelling action.

3. The injection device according to claim 1, further comprising wireless communication structure for transferring information regarding the occurrences of acceleration of the piston rod system detected by the integrated sensor during the dose expelling action to an external data receiving device.

4. The injection device according claim 1, wherein the piston rod system comprises a piston rod and a piston washer, the piston rod extending along a longitudinal axis from a proximal piston rod end to a distal piston rod end and the piston washer being arranged at the distal piston rod end, and
wherein the integrated sensor is integrated in the piston washer.

5. The injection device according to claim 4, wherein the integrated sensor is a force sensor arranged to measure the force applied to the piston washer by the piston rod.

6. The injection device according to claim 5, wherein the piston washer comprises a piston rod bearing structure comprising an interface sheet adapted to receive the distal piston rod end, the interface sheet extending transversally to the longitudinal axis and exhibiting axial resilience, and
wherein the force sensor is a strain or stress responsive sensor arranged on the interface sheet.

7. The injection device according to claim 5,
wherein the interface sheet has a first bending stiffness,
wherein the piston rod bearing structure further comprises an annular spacer, and a supporting plate having a predetermined second bending stiffness which is larger than the first bending stiffness, the supporting plate being sandwiched between the annular spacer and the interface sheet, and
wherein the distal piston rod end is arranged to abut a portion of the interface sheet which is supported by the supporting plate but unsupported by the annular spacer.

8. The injection device according to claim 7,
wherein the piston washer further comprises an electric power supply element arranged to support the annular spacer, and
wherein the interface sheet forms part of a foil member also comprising a bottom sheet arranged in contact with a surface of the electric power supply element opposite the annular spacer, and a connecting portion connecting the interface sheet and the bottom sheet, the foil member further carrying the processor and comprising printed electric leads connecting the strain or stress responsive sensor and the processor.

9. The injection device according to claim 7, wherein the strain or stress responsive sensor comprises a piezoelectric sensor printed on a surface portion of the interface sheet which faces the supporting plate.

10. The injection device according to claim 4, wherein the integrated sensor is a pressure sensor arranged in fluid communication with the medical substance.

11. The injection device according to claim 10, wherein the piston washer comprises a hollow structure arranged to extend through the piston and into an interior of the cartridge, and
wherein the pressure sensor is arranged in fluid communication with an interior of the hollow structure.

12. The injection device according to claim 11, wherein the piston washer further comprises
a piston rod bearing surface adapted to receive the distal piston rod end,
a piston interface layer adapted to interact with the piston,
a hub member carrying the hollow structure, the hollow structure extending through the piston interface layer,
an electric power supply element arranged between the piston rod bearing surface and the hub member, and
a foil member comprising a top sheet arranged between the piston rod bearing surface and the electric power supply element, a bottom sheet arranged between the electric power supply element and the hub member and carrying the pressure sensor, and a connecting portion connecting the top sheet and the bottom sheet, the foil member further carrying the processor and comprising printed electric leads connecting the pressure sensor and the processor.

13. The injection device according to claim 4, wherein the integrated sensor is a pressure sensor arranged in fluid communication with a fluid filled hollow of the piston.

14. The injection device according to claim 13, wherein the piston washer comprises a hollow structure arranged to extend into the fluid filled hollow of the piston, and
wherein the pressure sensor is arranged in fluid communication with an interior of the hollow structure.

15. The injection device according to claim 14, wherein the piston washer further comprises
a piston rod bearing surface adapted to receive the distal piston rod end,
a piston interface layer adapted to interact with the piston,
a hub member carrying the hollow structure, the hollow structure extending through the piston interface layer,
an electric power supply element arranged between the piston rod bearing surface and the hub member, and
a foil member comprising a top sheet arranged between the piston rod bearing surface and the electric power supply element, a bottom sheet arranged between the electric power supply element and the hub member and carrying the pressure sensor, and a connecting portion connecting the top sheet and the bottom sheet, the foil member further carrying the processor and comprising printed electric leads connecting the pressure sensor and the processor.
